(19) United States
(12) Patent Application Publication
(10) Pub. No.: US 2026/0053425 A1
(43) Pub. Date: Feb. 26, 2026

(54) SICKNESS ASSESSMENT SYSTEM AND SICKNESS ASSESSMENT METHOD

(71) Applicants: National Institute of Information and Communications Technology, Tokyo (JP); HONDA MOTOR CO., LTD., Tokyo (JP)

(72) Inventors: Hiroshi Ban, Koganei-shi (JP); Takatsugu Aihara, Koganei-shi (JP); Sachiko Yamada, Tokyo (JP)

(21) Appl. No.: 19/304,631

(22) Filed: Aug. 20, 2025

(30) Foreign Application Priority Data
Aug. 23, 2024 (JP) 2024-143192

(51) Int. Cl. A61B 5/00; A61B 5/11; A61B 5/378; A61B 5/384

(52) U.S. Cl. CPC A61B 5/4023; A61B 5/1103; A61B 5/1104; A61B 5/378; A61B 5/384; A61B 5/7425; A61B 2503/12

(57) ABSTRACT

A sickness assessment system includes a video storage unit configured to store video data that causes a visually induced self-motion sensation in a subject, a pattern storage unit configured to store an obstruction pattern that obstructs a partial area of a video based on the video data, a display control unit configured to display, on a display unit, an assessment video in which the obstruction pattern stored in the pattern storage unit is superimposed on the video based on the video data stored in the video storage unit, and an assessment processing unit configured to assess sickness of the subject on the basis of output information output from the subject who has viewed the displayed assessment video.

### CROSS-REFERENCE TO RELATED APPLICATION

This application is based upon and claims the benefit of priority from Japanese Patent Application No. 2024-143192, filed on Aug. 23, 2024, the entire content of which is incorporated herein by reference.

### BACKGROUND OF THE INVENTION

### Field of the Invention

The present invention relates to a sickness assessment system and a sickness assessment method.

### Description of Related Art

In recent years, a technology for assessing sickness using a displayed image has become known (refer to, for example, Patent Document 1). Meanwhile, in conventional sickness assessment methods, for example, when a motion sickness based on a field of view from a vehicle such as a car is assessed, it has been common to assess the sickness by having a subject board an actual vehicle or by using an image captured when the subject has actually boarded a vehicle.

### PATENT DOCUMENTS

[Patent Document 1] Japanese Unexamined Patent Application, First Publication No. 2013-197771

### SUMMARY OF THE INVENTION

However, the conventional methods described above require the use of an actual vehicle for the assessment, and only limited assessments can be performed.

The present invention has been made to solve the problems described above, and an object thereof is to provide a sickness assessment system and a sickness assessment method that can perform a highly flexible sickness assessment without using an actual vehicle.

To solve the problems described above, one aspect of the present invention provides a sickness assessment system including a video storage unit configured to store video data that causes a visually induced self-motion sensation in a subject, a pattern storage unit configured to store an obstruction pattern that obstructs a partial area of a video based on the video data, a display control unit configured to display, on a display unit, an assessment video in which the obstruction pattern stored in the pattern storage unit is superimposed on the video based on the video data stored in the video storage unit, and an assessment processing unit configured to assess sickness of the subject on the basis of output information output from the subject who has viewed the displayed assessment video.

In addition, another aspect of the present invention provides a sickness assessment method for a sickness assessment system including a video storage unit storing video data that causes a visually induced self-motion sensation in a subject, and a pattern storage unit storing an obstruction pattern for obstructing a partial area of a video based on the video data, the sickness assessment method including a display control step in which a display control unit displays, on a display unit, an assessment video in which the obstruction pattern stored in the pattern storage unit is superimposed on the video based on the video data stored in the video storage unit, and an assessment step in which an assessment processing unit assesses sickness of the subject on the basis of output information output from the subject who has viewed the displayed assessment video.

According to the present invention, a highly flexible sickness assessment can be performed without using an actual vehicle.

### BRIEF DESCRIPTION OF THE DRAWINGS

FIG. 1 is a functional block diagram showing an example of a sickness assessment system according to a first embodiment.

FIG. 2 is a diagram showing an example of an optical flow video in the first embodiment.

FIG. 3 is a diagram showing an example of an obstruction pattern in the first embodiment.

FIG. 4 is a diagram showing an example of an assessment video generated by a display control unit in the first embodiment.

FIG. 5 is a flowchart showing an example of an operation of the sickness assessment system according to the first embodiment.

FIG. 6 is a diagram showing an example of an assessment result of the sickness assessment system according to the first embodiment.

FIG. 7 is a functional block diagram showing an example of a sickness assessment system according to a second embodiment.

### DETAILED DESCRIPTION OF THE INVENTION

A sickness assessment system and a sickness assessment method according to one embodiment of the present invention will be described below with reference to the drawings.

### First Embodiment

FIG. 1 is a functional block diagram showing an example of a sickness assessment system 1 according to a first embodiment.

As shown in FIG. 1, the sickness assessment system 1 includes a sickness assessment device 10, a display unit 20, an input unit 30, and a measurement unit 40.

The sickness assessment system 1 according to the present embodiment performs a sickness assessment (vehicle sickness assessment) using obstructions (for example, window frames, pillars, seat headrests, and the like) based on a design of a vehicle (mobility) such as a car.

The display unit 20 is, for example, a display device such as a liquid crystal display, and displays an assessment video for the vehicle sickness assessment of a subject TU. The display unit 20 may be, for example, capable of displaying a three-dimensional video that the subject TU can view in a stereoscopic manner using 3D glasses.

The input unit 30 is, for example, an input device such as a keyboard, a mouse, or an operation pad, and receives assessment information indicating, for example, a level of sickness of the subject TU. The assessment information may be information indicating a sickness rank, such as “rank 1” to “rank 5,” or may be information comparing two assessment videos and indicating which one is more likely to cause sickness. The input unit 30 transmits the assessment information input by the subject TU to the sickness assessment device 10.

The measurement unit 40 is, for example, an electroencephalograph that measures a brain activity, magnetic resonance imaging (MRI), magnetoencephalography (MEG) that measures magnetoencephalograms, a camera (imaging device) that measures an eye movement, a body movement, and subjective reports of psychological indicators (nausea, dizziness, and the like), or the like. The measurement unit 40 measures physiological parameters of the subject TU, including at least any one of an eye movement, a brain activity, a body movement, and subjective reports of psychological indicators (nausea, dizziness, and the like), as output information output from the subject TU that has viewed a displayed assessment video. Here, the physiological parameters include, for example, information indicating a brain activity (MRI, electroencephalogram, magnetoencephalogram, and the like), an eye movement, a body movement, and subjective reports of psychological indicators (nausea, dizziness, and the like). The measurement unit 40 transmits the measured physiological parameters to the sickness assessment device 10.

The sickness assessment device 10 is, for example, a computer device such as a personal computer or a server device. The sickness assessment device 10 includes a storage unit 11 and a control unit 12.

The storage unit 11 stores various types of information used for various types of processing executed by the sickness assessment device 10. The storage unit 11 includes a video storage unit 111, a pattern storage unit 112, an output information storage unit 113, and an assessment result storage unit 114.

The video storage unit 111 stores video data (moving image data) that causes a visually induced self-motion sensation in the subject TU. Here, the video data is, for example, video data including an optical flow video that uses movement of dots to cause a visually induced self-motion sensation. The optical flow video can be, for example, an enlarged pattern in which dots move from a center of the video (moving image) to the periphery, or a pattern in which dots move from the center of the image (moving image) to the periphery and slalom.

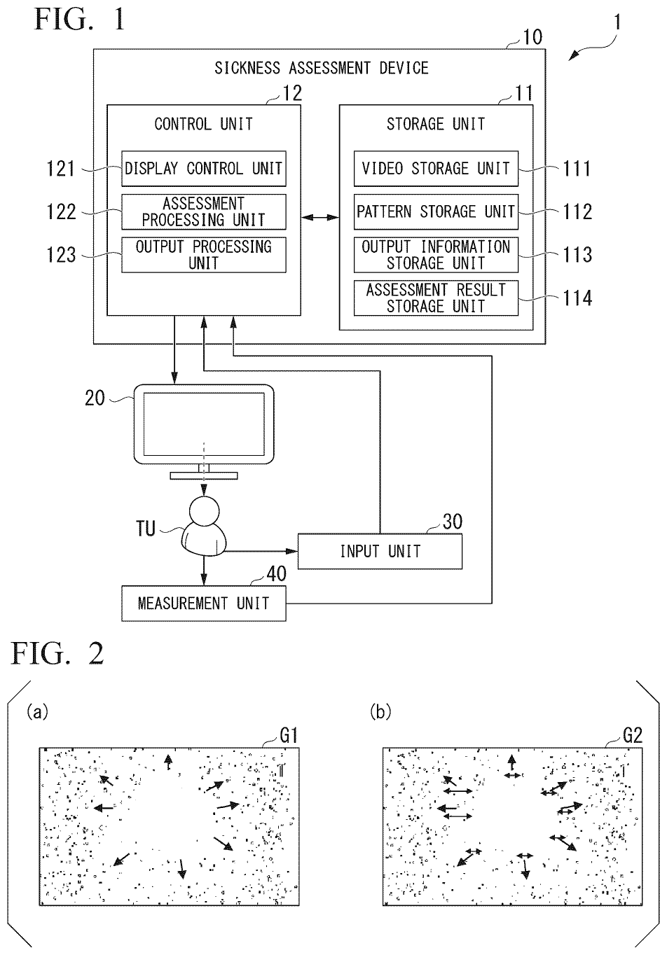

Here, with reference to FIG. 2, an example of an optical flow video (moving image) used by the sickness assessment system 1 will be described.

FIG. 2 is a diagram showing an example of an optical flow video in the present embodiment.

An image G1 shown in (a) of FIG. 2 shows an optical flow video of an enlarged pattern. As shown in the image G1, the video gives an illusion that the subject TU riding on the vehicle is moving forward by moving dots from the center to the periphery in the optical flow video of the enlarged pattern.

An image G2 shown in (b) of FIG. 2 shows an optical flow video of a slalom pattern. As shown in the image G2, in the optical flow video of the slalom pattern, dots move from the center to the periphery and also move left and right to cause a slalom, creating an illusion that the subject TU riding on the vehicle is moving forward while swaying left and right.

Note that in the example shown in FIG. 2, black dots are shown moving on a white screen to make the description easier to understand, but in actual use, white dots (glowing dots) may be shown moving on a black screen. The size of the dots may also be made larger as they move to the periphery.

In addition, the video storage unit 111 stores a plurality of pieces of video data as shown in FIG. 2 for comparison. For example, the video storage unit 111 stores a video ID (video identification information) that identifies a video in association with the video data.

Returning to the description of FIG. 1, the pattern storage unit 112 stores an obstruction pattern that obstructs a partial area of the video based on the video data. The obstruction pattern is, for example, a pattern that indicates a shape of an obstruction based on a design of the vehicle, which obstructs the outside view when the subject TU looks outside from inside the vehicle. In other words, the obstruction pattern is, for example, a pattern that imitates the obstruction of a field of view caused by pillars, window frames, headrests of seats, or the like.

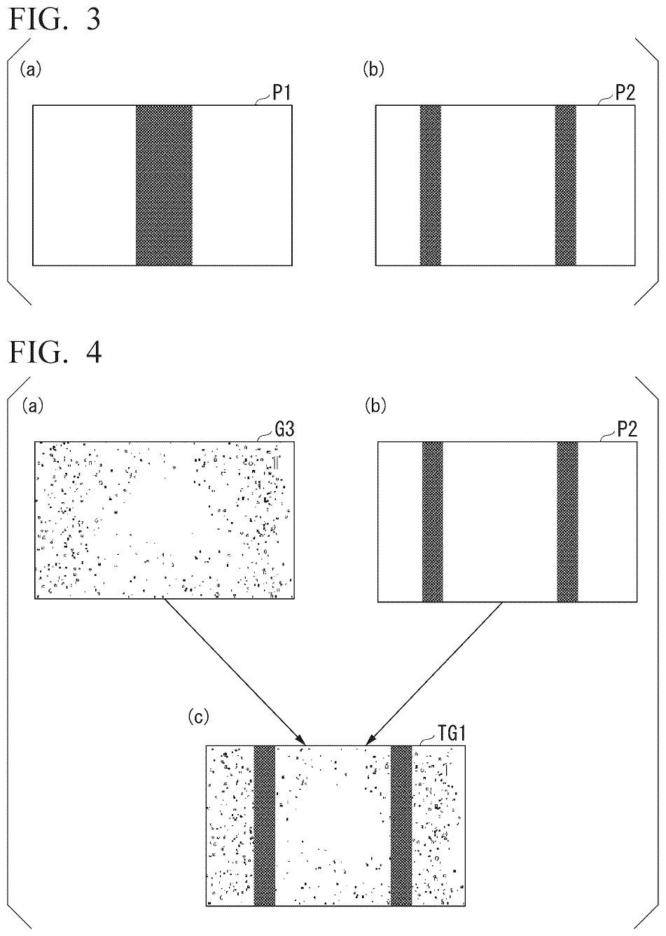

Here, an example of an obstruction pattern used by the sickness assessment system 1 will be described with reference to FIG. 3.

FIG. 3 is a diagram showing an example of an obstruction pattern in the present embodiment.

An obstruction pattern P1 shown in (a) of FIG. 3 shows a pattern with an obstruction in the center (middle part) of the screen. The obstruction pattern P1 is, for example, a pattern that imitates a headrest from a back seat of a car to the front seat.

An obstruction pattern P2 shown in (b) of FIG. 3 shows a pattern with two obstructions on the left and right of the screen. The obstruction pattern P2 is, for example, a pattern that imitates a pillar in a front part of the car.

The pattern storage unit 112 stores a plurality of obstruction patterns such as those shown in FIG. 3 for comparison. The pattern storage unit 112 stores, for example, a pattern ID (pattern identification information) that identifies an obstruction pattern and the obstruction pattern in association with each other.

Returning to the description of FIG. 1, the output information storage unit 113 stores output information output from the subject TU that has viewed an assessment video. The output information is, for example, physiological parameters measured by the measurement unit 40 (information indicating a brain activity (MRI, electroencephalogram, magnetoencephalogram, or the like), an eye movement, a body movement, and subjective reports of psychological indicators (nausea, dizziness, and the like)), or assessment information indicating a level of sickness input by the subject TU from the input unit 30. The output information storage unit 113 stores, for example, information indicating the type of output information, a subject ID (subject identification information) that identifies the subject TU, a video ID, a pattern ID, and the output information in association with each other.

The assessment result storage unit 114 stores an assessment result indicating a result of a sickness assessment. The assessment result storage unit 114 stores, for example, a subject ID, a video ID, a pattern ID, and an assessment result in association with each other.

The control unit 12 is, for example, a processor including a central processing unit (CPU) and the like, and controls the sickness assessment device 10 in an integrated manner. The control unit 12 executes various types of processing executed by the sickness assessment device 10. In addition, the control unit 12 includes a display control unit 121, an assessment processing unit 122, and an output processing unit 123.

The display control unit 121 displays an assessment video on the display unit 20, in which an obstruction pattern stored in the pattern storage unit 112 is superimposed on a video based on video data stored in the video storage unit 111. The display control unit 121 acquires video data from the video storage unit 111, acquires an obstruction pattern from the pattern storage unit 112, and generates, for example, an assessment video in which the obstruction pattern is superimposed on a video based on the video data, as shown in FIG. 4. That is, the display control unit 121 generates an assessment video in which the obstruction pattern is superimposed on a video based on the video data, so that the obstruction pattern blocks (obstructs) a partial area of the video based on the video data.

FIG. 4 is a diagram showing an example of an assessment video generated by the display control unit 121 in the present embodiment.

An image G3 shown in (a) of FIG. 4 shows an example of the video based on video data, and (b) of FIG. 4 shows the obstruction pattern P2. An image TG1 shown in (c) of FIG. 4 shows an image example of the assessment video.

As shown in FIG. 4, the display control unit 121 generates an assessment video such as the image TG1 shown in (c) of FIG. 4 by superimposing the obstruction pattern P2 shown in (b) of FIG. 4 on the video (image G3) shown in FIG. 4(a). The display control unit 121 displays the generated assessment video on the display unit 20 so that the subject TU can view it.

The display control unit 121 may display the assessment video on the display unit 20 as a three-dimensional video that the subject TU can view in a stereoscopic manner. For example, the display control unit 121 may generate the assessment video as a 3D video and display it on the display unit 20 so that the subject TU can view it in stereoscopic form, for example, by wearing 3D glasses.

The assessment processing unit 122 assesses the sickness of the subject TU on the basis of output information output from the subject TU who has viewed the assessment video displayed on the display unit 20. The assessment processing unit 122 assesses the sickness of the subject TU on the basis of, for example, the physiological parameters measured by the measurement unit 40, or the assessment information indicating the level of sickness input by the subject TU from the input unit 30.

The assessment processing unit 122 stores the acquired physiological parameters and assessment information as output information in the output information storage unit 113. The assessment processing unit 122 stores, in the output information storage unit 113, for example, information indicating the type of output information, the subject ID (subject identification information) for identifying the subject TU, the video ID, the pattern ID, and the output information in association with each other.

The assessment processing unit 122 also executes a sickness assessment of the subject TU using the physiological parameters and assessment information stored in the output information storage unit 113, either alone or in combination. Note that physiological parameters such as a brain activity (MRI, electroencephalogram, magnetoencephalogram, or the like), an eye movement, and a body movement are known to change depending on conditions such as motion sickness, and the assessment processing unit 122 performs a sickness assessment using these existing technologies.

The assessment processing unit 122 may execute, for example, an assessment by comparing two obstruction patterns or two videos to see which is more likely to cause sickness, or may execute an assessment by combining a plurality of pieces of output information to calculate a score indicating likelihood of causing sickness. The assessment processing unit 122 may also rank the plurality of obstruction patterns or a plurality of videos in terms of how likely they are to cause sickness on the basis of, for example, the calculated score or the like.

The assessment processing unit 122 stores the assessment result in the assessment result storage unit 114. The assessment processing unit 122 may store, for example, a subject ID, a video ID, a pattern ID, and the assessment result in the assessment result storage unit 114 in association with each other. Furthermore, the assessment processing unit 122 may store, as the assessment results, a result of comparison between two obstruction patterns or two videos, and rankings of the plurality of obstruction patterns or the plurality of videos in the assessment result storage unit 114.

The output processing unit 123 outputs the assessment results assessed by the assessment processing unit 122 to the outside. For example, the output processing unit 123 may aggregate the assessment results stored in the assessment result storage unit 114 and output them to the outside, or may generate display information based on the assessment results stored in the assessment result storage unit 114 and output the display information to the outside.

Next, an operation of the sickness assessment system 1 according to the present embodiment will be described with reference to the drawings.

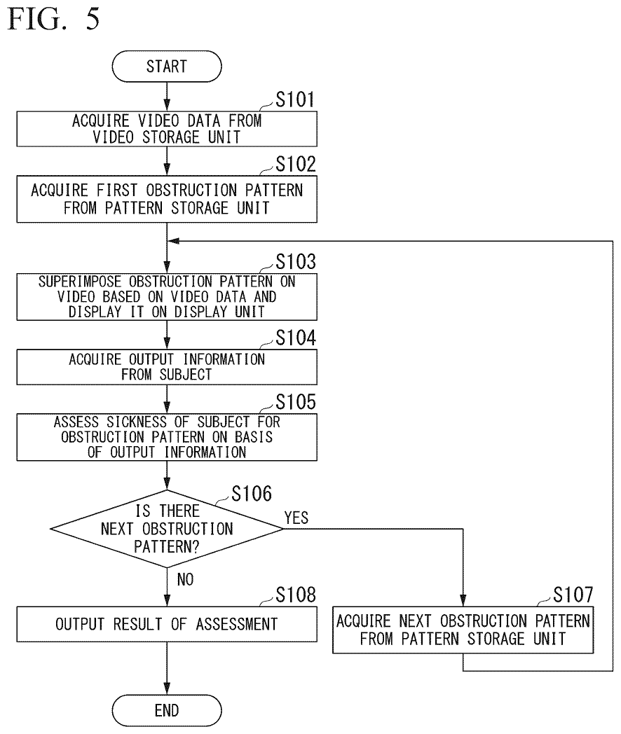

FIG. 5 is a flowchart showing an example of the operation of the sickness assessment system 1 according to the present embodiment. An example shown in FIG. 5 describes an example of performing sickness assessment for the plurality of obstruction patterns.

As shown in FIG. 5, the sickness assessment device 10 of the sickness assessment system 1 first acquires video data from the video storage unit 111 (step S101). The display control unit 121 of the sickness assessment device 10 acquires, for example, video data of a video as shown in FIG. 2 from the video storage unit 111.

Next, the display control unit 121 acquires a first obstruction pattern from the pattern storage unit 112 (step S102). The display control unit 121 acquires, for example, obstruction data as shown in FIG. 3 from the pattern storage unit 112.

Next, the display control unit 121 superimposes an obstruction pattern on a video based on the video data and displays it on the display unit 20 (step S103). The display control unit 121 generates an assessment video in which the obstruction pattern is superimposed on the video, for example, as shown in FIG. 3, and displays the generated assessment video on the display unit 20.

Next, the assessment processing unit 122 of the sickness assessment device 10 acquires output information from the subject TU (step S104). As output information, the assessment processing unit 122 acquires the physiological parameters measured by the measurement unit 40 and the assessment information input by the subject TU from the input unit 30. In addition, the assessment processing unit 122 stores, for example, information indicating the type of output information, the subject ID, the video ID, the pattern ID, and the output information in the output information storage unit 113 in association with each other.

Next, the assessment processing unit 122 assesses the sickness of the subject for an obstruction pattern on the basis of the output information (step S105). The assessment processing unit 122 assesses the sickness of the subject TU on the basis of, for example, the physiological parameters measured by the measurement unit 40 or the assessment information input by the subject TU from the input unit 30, which are stored in the output information storage unit 113. The assessment processing unit 122 stores a result of the assessment in the assessment result storage unit 114.

Next, the control unit 12 of the sickness assessment device 10 determines whether there is a next obstruction pattern (step S106). The control unit 12 determines whether there is a next obstruction pattern, for example, based on whether the number of obstruction patterns has reached a pre-specified number of patterns or whether a last obstruction pattern stored in the pattern storage unit 112 has been reached. When there is a next obstruction pattern (YES in step S106), the control unit 12 advances the processing to step S107. When there is no next obstruction pattern (NO in step S106), the control unit 12 advances the processing to step S108.

In step S107, the display control unit 121 acquires a next obstruction pattern from the pattern storage unit 112. After processing of step S107, the display control unit 121 returns the processing to step S103.

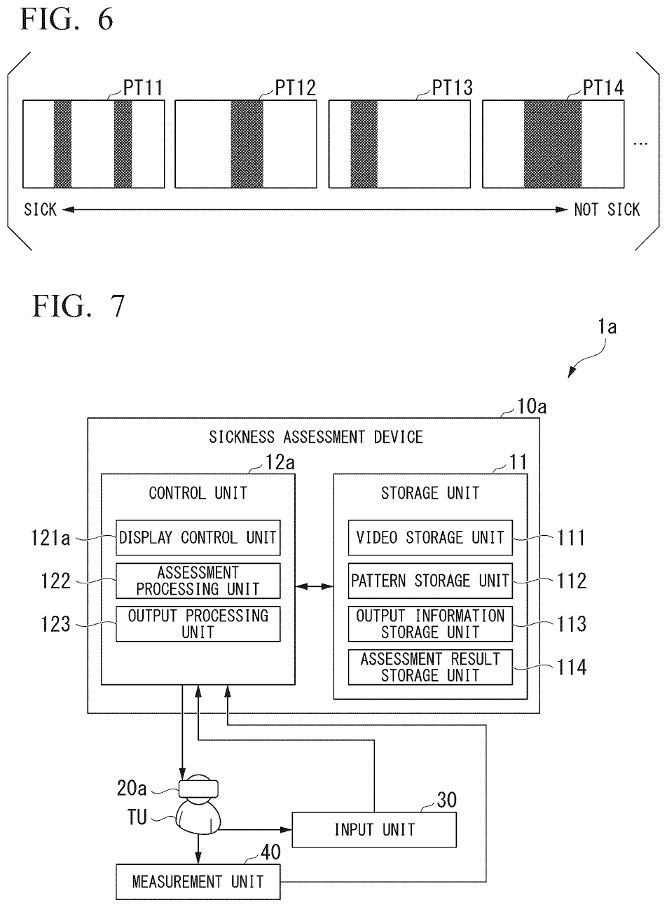

In step S108, the output processing unit 123 of the control unit 12 outputs the assessment result. The output processing unit 123 generates, for example, display information in which the likelihood of causing sickness is arranged in order using the plurality of obstruction patterns, as shown in FIG. 6, and outputs the display information to the outside. After processing of step S108, the control unit 12 ends the processing.

FIG. 6 is a diagram showing an example of an assessment result of the sickness assessment system 1 according to the present embodiment.

The example shown in FIG. 6 is an example in which the sickness assessment device 10 outputs an assessment result in which the likelihood of causing sickness is arranged in order using the plurality of obstruction patterns as the assessment result.

In the example shown in FIG. 6, the assessment processing unit 122 outputs display information that displays, from left to right, in order of an obstruction pattern that is most likely to cause sickness to an obstruction pattern that is least likely to cause sickness. That is, the assessment results shown in FIG. 6 show that an obstruction pattern PT11 is most likely to cause sickness, followed by an obstruction pattern PT12, an obstruction pattern PT13, and an obstruction pattern PT14, in that order.

In the example shown in FIGS. 5 and 6, an example has been described in which the sickness assessment system 1 displays rankings of the likelihood of causing sickness among the plurality of obstruction patterns as the assessment result, but the present invention is not limited to this, and it may perform assessment by comparing two obstruction patterns or two videos, or simply by assessing the sickness of the subject TU for one assessment video.

As described above, the sickness assessment system 1 according to the present embodiment includes a video storage unit 111, a pattern storage unit 112, a display control unit 121, and an assessment processing unit 122. The video storage unit 111 stores video data that causes a visually induced self-motion sensation in the subject TU. The pattern storage unit 112 stores an obstruction pattern that obstructs a partial area of the video based on the video data. The display control unit 121 displays an assessment video on the display unit 20, which has a video based on the video data stored in the video storage unit 111 superimposed with an obstruction pattern stored in the pattern storage unit 112. The assessment processing unit 122 assesses the sickness of the subject TU on the basis of the output information output from the subject TU that has viewed the displayed assessment video.

As a result, the sickness assessment system 1 according to the present embodiment superimposes the obstruction pattern that obstructs a partial area of the video onto the video that causes a visually induced self-motion sensation in the subject TU, and displays a result on the display unit 20. Therefore, for example, by changing the obstruction pattern, a highly flexible sickness assessment can be performed without using an actual vehicle. The sickness assessment system 1 according to the present embodiment can develop a design that is less likely to cause sickness in vehicles such as cars.

The sickness assessment system 1 according to the present embodiment can assess the likelihood of causing sickness in advance when, for example, pillars, seats, cockpits, and the like are designed to design a field of view from a rear seat of a car, which is less likely to cause sickness. In addition, by changing a parameter of the optical flow, the sickness assessment system 1 according to the present embodiment can assess the design and the likelihood of causing sickness of various vehicles that induce sickness, such as ship sickness and airplane sickness, not limited to movement of a car.

In the present embodiment, the video data includes an optical flow video that uses movement of dots to cause a visually induced self-motion sensation. In addition, the obstruction pattern is a pattern that represents a shape of an obstruction based on the design of the vehicle that obstructs an outside view when the subject TU looks outside from inside the vehicle.

As a result, the sickness assessment system 1 according to the present embodiment can easily change the assessment video by using the optical flow video. Furthermore, the sickness assessment system 1 according to the present embodiment can assess the likelihood of causing sickness in advance when the shape of an obstruction is designed by using a pattern indicating the shape of the obstruction based on the design of the vehicle as an obstruction pattern.

Moreover, in the present embodiment, the display control unit 121 displays the assessment video on the display unit 20 as a three-dimensional video that the subject TU can view in a stereoscopic manner. For example, the display control unit 121 generates a three-dimensional assessment video that can be viewed in a stereoscopic manner using 3D glasses, and displays it on the display unit 20, and the subject TU wears the 3D glasses and views the assessment video.

As a result, the sickness assessment system 1 according to the present embodiment can assess the likelihood of causing sickness more realistically and accurately by using a three-dimensional video that can be viewed in a stereoscopic manner as the assessment video.

Furthermore, the sickness assessment system 1 according to the present embodiment includes a measurement unit 40 that measures the physiological parameters of the subject TU including at least any one of an eye movement, a brain activity, a body movement, and subjective reports of psychological indicators (for example, nausea, dizziness, and the like) as output information. The assessment processing unit 122 assesses the sickness of the subject TU on the basis of the physiological parameters measured by the measurement unit 40 or the assessment information indicating the level of sickness input by the subject TU from the input unit 30.

As a result, the sickness assessment system 1 according to the present embodiment can assess the sickness of the subject TU more accurately and objectively by using the physiological parameters of the subject TU including at least any one of an eye movement, a brain activity, a body movement, and subjective reports of psychological indicators (for example, nausea, dizziness, and the like). In addition, the sickness assessment system 1 according to the present embodiment can assess a subjective sickness of the subject TU by using the assessment information indicating the level of sickness input by the subject TU from the input unit 30.

The sickness assessment method according to the present embodiment is a sickness assessment method of the sickness assessment system 1 including the video storage unit 111 that stores video data that causes a visually induced self-motion sensation in the subject TU, and the pattern storage unit 112 that stores an obstruction pattern that obstructs a partial area of the video based on the video data, and includes a display control step and an assessment step. In the display control step, the display control unit 121 displays an assessment video on the display unit 20, which has a video based on the video data stored in the video storage unit 111 superimposed with an obstruction pattern stored in the pattern storage unit 112. In the assessment step, the assessment processing unit 122 assesses the sickness of the subject TU on the basis of output information output from the subject TU that has viewed the displayed assessment video.

As a result, the sickness assessment method according to the present embodiment has the same effect as the sickness assessment system 1 described above. For example, by changing the obstruction pattern, it is possible to perform a highly flexible sickness assessment without using an actual vehicle.

### Second Embodiment

Next, a sickness assessment system 1a according to a second embodiment will be described with reference to the drawings.

Note that a modified example in which a head-mounted display 20a is used for the display unit 20 will be described using the sickness assessment system 1a according to the present embodiment.

FIG. 7 is a functional block diagram showing an example of the sickness assessment system 1a according to the second embodiment.

As shown in FIG. 7, the sickness assessment system 1a includes a sickness assessment device 10a, a head-mounted display 20a, an input unit 30, and a measurement unit 40.

Note that in FIG. 7, the same constituents as those in FIG. 1 are given the same reference numerals and their description will be omitted.

The head-mounted display 20a is a display device that can be held on a head of the subject TU, and is capable of displaying three-dimensional video that the subject TU can view in a stereoscopic manner, such as a virtual reality (VR) video. The head-mounted display 20a displays the VR video output from the sickness assessment device 10a.

The sickness assessment device 10a is, for example, a computer device such as a personal computer or a server device, and includes a storage unit 11 and a control unit 12a.

The control unit 12a is, for example, a processor including a CPU and the like, and controls the sickness assessment device 10a in an integrated manner. The control unit 12a includes a display control unit 121a, an assessment processing unit 122, and an output processing unit 123.

The display control unit 121a has the same basic functions as the display control unit 121 of the first embodiment, but generates a VR video for the head-mounted display 20a as an assessment video, outputs it to the head-mounted display 20a for display. In other words, the display control unit 121a generates a three-dimensional video that the subject TU can view in a stereoscopic manner as an assessment video, and displays it on the head-mounted display 20a.

As described above, in the sickness assessment system 1a of the present embodiment, the display unit 20 is a head-mounted display 20a that can be held on the head of the subject TU, and the display control unit 121a generates a three-dimensional video that the subject TU can view in a stereoscopic manner as an assessment video, and displays it on the head-mounted display 20a.

As a result, the sickness assessment system 1a according to the present embodiment can assess the sickness of the subject TU using an assessment video that is closer to reality, for example, by displaying a VR video on the head-mounted display 20a.

Note that the present invention is not limited to the embodiments described above, and can be modified within a range not departing from the gist of the present invention.

For example, in each of the embodiments described above, an example has been described in which the sickness assessment system 1 (1a) assesses motion sickness, but the present invention is not limited to this, and the system may also be used to assess, for example, a design which is unlikely to cause sickness for video content such as a browser, a web page, a display, and the like.

In each of the embodiments described above, an example has been described in which the display control unit 121 (121a) may use an optical flow video as the assessment video, but the present invention is not limited to this and other videos may also be used.

In each of the embodiments described above, an example has been described in which the display control unit 121 (121a) uses an enlarged pattern of a video and a slalom video when an optical flow is used, but the present invention is not limited to this and other optical flow videos may also be used.

In addition, in each of the embodiments described above, an example has been described in which the physiological parameters are, for example, information indicating a brain activity (MRI, electroencephalogram, magnetoencephalogram, or the like), an eye movement, a body movement, and subjective report of psychological indicators (nausea, dizziness, and the like), but the present invention is not limited to this and other physiological parameters may also be used.

In addition, in each of the embodiments described above, an example has been described in which the sickness assessment device 10 (10a) is realized by a single device, but the present invention is not limited to this, and may also be realized by a plurality of devices (for example, a plurality of server devices). Moreover, for example, part of the storage unit 11 may be provided in an external device (external server device, or the like).

Each constituent of the sickness assessment system 1 (1a) described above has a computer system inside. Then, a program for realizing the function of each constituent of the sickness assessment system 1 (1a) described above may be recorded on a computer-readable recording medium, and the program recorded on this recording medium may be read into the computer system and executed to perform processing in each constituent of the sickness assessment system 1 (1a) described above. Here, “reading a program recorded on a recording medium into the computer system and executing it” includes installing the program into the computer system. It is assumed that the “computer system” referred to herein includes an OS and hardware such as peripheral devices.

In addition, the “computer system” may include a plurality of computer devices connected via a network, including communication lines such as the Internet, WAN, LAN, and dedicated lines. In addition, a “computer-readable recording medium” refers to a portable medium such as a flexible disk, an optical magnetic disc, an ROM, and a CD-ROM, and a storage device such as a hard disk built into the computer system. In this manner, the recording medium storing the program may be a non-transitory recording medium such as a CD-ROM.

In addition, the recording medium also includes an internal or external recording medium, which is accessible from a distribution server to distribute the program. Note that the program may be divided into a plurality of parts, downloaded at different timings, and then combined in each constituent of the sickness assessment system 1 (1a), or each of the divided programs may be distributed by a different distribution server. In addition, it is assumed that the “computer-readable recording medium” includes a medium that holds a program for a certain period of time, such as an internal volatile memory (RAM) of the computer system, which serves as a server or client when the program is transmitted via a network. In addition, the program may also be for realizing part of the functions described above. Furthermore, the program may be a so-called differential file (differential program) that can realize the functions described above in combination with a program already recorded in the computer system.

Part or all of the functions described above may be realized as an integrated circuit such as large scale integration (LSI). Each of the functions described above may be individually implemented as a processor, or part or all of the functions may be integrated into a processor. In addition, the integrated circuit implementation method is not limited to LSI, and may be realized using a dedicated circuit or a general-purpose processor. Furthermore, when an integrated circuit implementation technology that replaces LSI appears due to advances in semiconductor technology, an integrated circuit using that technology may also be used.

What is claimed is:

1. A sickness assessment system comprising:
a video storage unit configured to store video data that causes a visually induced self-motion sensation in a subject;
a pattern storage unit configured to store an obstruction pattern that obstructs a partial area of a video based on the video data;
a display control unit configured to display, on a display unit, an assessment video in which the obstruction pattern stored in the pattern storage unit is superimposed on the video based on the video data stored in the video storage unit; and
an assessment processing unit configured to assess sickness of the subject on the basis of output information output from the subject who has viewed the displayed assessment video.

2. The sickness assessment system according to claim 1,
wherein the video data includes a video of an optical flow that uses a movement of dots to cause the visually induced self-motion sensation, and
wherein the obstruction pattern is a pattern that represents a shape of an obstruction based on a design of the vehicle that obstructs an outside view when the subject looks outside from inside the vehicle.

3. The sickness assessment system according to claim 2,
wherein the display control unit is configured to display the assessment video on the display unit as a three-dimensional video that is viewed stereoscopically by the subject.

4. The sickness assessment system according to claim 3,
wherein the display unit is a head-mounted display that is held on a head of the subject, and
wherein the display control unit is configured to generate a three-dimensional video that is viewed stereoscopically by the subject as the assessment video and display the video on the head-mounted display.

5. The sickness assessment system according to claim 1, further comprising:
a measurement unit configured to measure, as the output information, physiological parameters of the subject, including at least any one of an eye movement, a brain activity, a body movement, and a subjective report of a psychological indicator, and
the assessment processing unit is configured to assess a sickness of the subject on the basis of the physiological parameters measured by the measurement unit or assessment information indicating a level of sickness input by the subject from an input unit.

6. A sickness assessment method for a sickness assessment system including a video storage unit storing video data that causes a visually induced self-motion sensation in a subject, and a pattern storage unit storing an obstruction pattern for obstructing a partial area of a video based on the video data, the sickness assessment method comprising:
a display control step in which a display control unit displays, on a display unit, an assessment video in which the obstruction pattern stored in the pattern storage unit is superimposed on the video based on the video data stored in the video storage unit; and
an assessment step in which an assessment processing unit assesses a sickness of the subject on the basis of output information output from the subject who has viewed the displayed assessment video.
